(19) United States
(12) Patent Application Publication
(10) Pub. No.: US 2026/0272280 A1
(43) Pub. Date: Sep. 17, 2026

(54) CONVOLUTIONAL NEURAL NETWORK CLASSIFICATION OF PRESENCE OR ABSENCE OF DISEASE WITH ENDOSCOPIC OR LARYNGOSCOPIC VIDEO

(71) Applicant: Ohio State Innovation Foundation, Columbus, OH (US)

(72) Inventors: Samantha KRENING, Columbus, OH (US); Sachin JHAWAR, Columbus, OH (US); Ryan GIFFORD, Columbus, OH (US); Kyle VANKOEVERING, Columbus, OH (US)

(21) Appl. No.: 19/166,701

(22) PCT Filed: Mar. 20, 2024
(86) PCT No.: PCT/US2024/020759

§ 371 Date: Sep. 18, 2025

Related U.S. Application Data
(60) Provisional application No. 63/491,188, filed on Mar. 20, 2023.

(51) Int. Cl. A61B 1/267; A61B 1/00; G06N 20/00; G06T 7/00

(52) U.S. Cl. CPC A61B 1/267; A61B 1/000094; A61B 1/000096; A61B 1/00043; G06N 20/00; G06T 7/0012; G06T 2207/10068; G06T 2207/20081; G06T 2207/20084; G06T 2207/30096

(57) ABSTRACT

An exemplary method and system are disclosed for a machine learning application in head and neck cancers, using deep learning methods (e.g., transformer) to classify whether there is evidence of an indicator of a disease or the disease itself, e.g., oropharyngeal squamous cell carcinoma (OPSCC) in endoscopic or laryngoscopic video, e.g., Video Nasopharyngolaryngoscopy (NPL) to inform and focus attention of the clinician to the same. The exemplary method and system do not provide diagnostics of cancer but rather provide visual indicators of an anomaly or indicators in the endoscopic or laryngoscopic video, e.g., Video Nasopharyngolaryngoscopy (NPL), to be used in diagnostic by a clinician.

### RELATED APPLICATION

This PCT patent application claims priority to, and the benefit of, U.S. Provisional Patent Application No. 63/491,188, filed Mar. 20, 2023, which is incorporated by reference herein in its entirety.

### BACKGROUND

The current diagnostic standard for oropharyngeal squamous cell carcinoma (OPSCC) and other nose, throat, and airway cancers involves endoscopy, PET, and biopsy of suspicious lesions. However, current methods to identify OPSCC are difficult with endoscopy.

While machine learning (ML) has been an area of interest in medicine, including disease diagnosis, triage and prognostication, clinical decision-making, surgical planning, intra-operative assistance, and patient education, research in ML applications in detecting head neck cancers (HNCs) has been primarily based on multispectral narrow band imaging (mNBI).

There is a benefit to improving the identification of unknown primary tumors and local surveillance for head and neck cancers.

### SUMMARY

An exemplary method and system are disclosed for a machine learning application in head and neck cancers, using deep learning methods (e.g., transformer) to classify whether there is evidence of an indicator of a disease or a proxy of the disease, e.g., oropharyngeal squamous cell carcinoma (OPSCC) in endoscopic or laryngoscopic video, e.g., Video Nasopharyngolaryngoscopy (NPL) to inform and focus attention of the clinician to the same. The exemplary method and system do not provide diagnostics of cancer but rather provide visual indicators of an anomaly or indicators in the endoscopic or laryngoscopic video, e.g., Video Nasopharyngolaryngoscopy (NPL), to be used in diagnostic by a clinician. A commercial or clinical system can not diagnose a disease without a clinician.

The exemplary system and method, in being operable in evaluating mutiple frames across a video, can also be employed to evaluate functional pathologies associated with neck, head, throat conditions that are exhibited across multiple frames. Examples of functional pathologies can include swallowing, breathing, phonation (e.g., act of vocal cord coming together), other body movement/vibration. Functional pathologies can also include the presence or indication of paralysis, as well as aspiration, e.g., into the lungs or trachea in view of an action (e.g., swallowing, breathing, speaking, movements, etc.), as well as spitting. The exemplary system and method can be employed in conjunction with test protocols, e.g., to evaluate functional pathologies as the subject or patient is asked to swallow different types of liquids. In some embodiments, the exemplary system and method can be employed to evaluate disease states such as neuromuscular abnormalities, among others.

The exemplary method and system could be applied to 2D data such as video laryngoscopy or endoscopy as a diagnostic-assist tool, particularly in cases where the identification of a malignancy as evidenced by lesions, nodules, and/or vasculatures may not be readily apparent to the clinician. The exemplary method and system may be employed in the screening, during the treatment, and after treatment, e.g., of OPSCC and other head and neck cancers (HNC). In some embodiments, the exemplary method and system can be applied to 3D tomography data that are assessed in 2D frames in the CNN layers but as 3D data in a multi-head self-attention module that connects to the CNN layers.

The exemplary system and method can be used to provide consistent diagnostics by clinicians in providing assistance to normalize the different levels of experience possessed by the clinicians.

In an aspect, a method is disclosed comprising receiving an input endoscopic or laryngoscopic video (e.g., video endoscopy, video laryngoscopy, e.g., Video Nasopharyngolaryngoscopy); determining, via a processor, an output score associated with a presence of a disease state or condition, as indicated at least by a presence of lesion, nodule, and vasculature, using a trained neural network based on the endoscopic or laryngoscopic video; and outputting the output score or a label derived from the same, wherein the output score or label for the presence of the disease state or condition is presented in the input endoscopic or laryngoscopic video to direct attention to the same.

In some embodiments, the trained neural network is configured to determine one or more frames associated with the presence of a disease state or condition, and wherein the (i) output score or the label and the (ii) an identifier associated with the one or more frames are presented in the endoscopic or laryngoscopic video.

In some embodiments, the output score associated with the presence of a disease state or condition includes a presence of a functional pathology.

In some embodiments, a unique output score or label is generated for each frame or a portion of the frames of the input endoscopic or laryngoscopic video.

In some embodiments, a single output score or label is generated for the input endoscopic or laryngoscopic video.

In some embodiments, the trained AI model comprises (i) CNN layers having CNN nodes that evaluate individual frames of the input endoscopic or laryngoscopic video and (ii) multi-head self attention module configured to evaluate the individual frame collectively.

In some embodiments, the trained AI model is a transformer.

In some embodiments, the trained AI model further includes an attention weight module or an RNN configured to determine importance of weight associated with the CNN layers, wherein the CNN nodes are individually mapped to the individual frames, the method further including identifying one or more frames contributing to the output score associated with the presence of a disease state or condition.

In some embodiments, a predefined number of frames of the input endoscopic or laryngoscopic video are combined (e.g., 20 frames are averaged) to be provided to the trained neural network, wherein each frame of the input endoscopic or laryngoscopic video is inputted into a respective CNN.

In some embodiments, the trained neural network includes a plurality of stages of 2D CNN, each separated by a rectified linear unit (ReLU) activation.

In some embodiments, the trained neural network was trained with a training data set comprising endoscopic or laryngoscopic video and an indication of a nodule as a ground truth.

In some embodiments, the trained neural network was trained with a training data set comprising endoscopic or laryngoscopic video and an indication of a lesion as a ground truth.

In some embodiments, the trained neural network was trained with a training data set comprising endoscopic or laryngoscopic video and an indication of vasculature as a ground truth.

In some embodiments, the output score for lesion, nodule, and vasculature and an associated frame in the input endoscopic or laryngoscopic video are presented in real-time in association with the acquisition of the endoscopic or laryngoscopic video.

In some embodiments, the output score for lesion, nodule, and vasculature and an associated frame in the input endoscopic or laryngoscopic video are presented in following acquisition of the endoscopic or laryngoscopic video.

In another aspect, a system is disclosed comprising an analysis system having a processor and a memory having instructions stored thereon, wherein execution of the instructions by the processor causes the processor to: receive an input endoscopic or laryngoscopic video (e.g., video endoscopy, video laryngoscopy, e.g., Video Nasopharyngolaryngoscopy); determine, via a processor, an output score associated with a disease state or condition, as indicated by at least a presence of lesion, nodule, and vasculature, using a trained neural network based on the endoscopic or laryngoscopic video; and output the output score, wherein the output score or label derived therefrom is presented in the input endoscopic or laryngoscopic video via a graphical user interface to direct attention to the same.

In some embodiments, a predefined number of frames of the input endoscopic or laryngoscopic video are combined (e.g., 20 frames are averaged) to be provided to the trained neural network, wherein each frame of the input endoscopic or laryngoscopic video is inputted into a respective CNN.

In some embodiments, the trained neural network includes a plurality of stages of 2D CNN, each separated by a rectified linear unit (ReLU) activation.

In some embodiments, the system further includes an endoscopic or laryngoscopic equipment configured to acquire the input endoscopic or laryngoscopic video.

In some embodiments, the trained neural network was trained with a training data set comprising endoscopic or laryngoscopic video and an indication of a nodule, a lesion, and/or vasculature as a ground truth.

In another aspect, a non-transitory computer-readable medium is disclosed having instructions stored thereon, wherein execution of the instructions by a processor causes the processor to receive an input endoscopic or laryngoscopic video (e.g., video endoscopy, video laryngoscopy, e.g., Video Nasopharyngolaryngoscopy); determine an output score associated with a disease state or condition, as indicated by at least a presence of lesion, nodule, and vasculature, using a trained neural network based on the endoscopic or laryngoscopic video; and output the output score or a label derived therefrom, wherein the output score is presented in the input endoscopic or laryngoscopic video via a graphical user interface to direct attention to the same.

### BRIEF DESCRIPTION OF DRAWINGS

FIGS. 1A-1C depict example model architectures with shared convolutions across frames and a self-attention mechanism to extract frame wise dependencies.

FIG. 2 depicts an example method in accordance with the present disclosure.

FIG. 3A depicts example computer instructions for a multi-head dot-product self attention module.

FIG. 3B depicts example computer instructions for training of the multi-head dot-product self attention module of FIG. 3A.

FIG. 3C depicts example computer instructions for an attention weight module.

FIGS. 4A-4B depict examples of lesions and nodules that may be found in neck, throat, or head region of a subject or patient.

FIG. 4C shows a photo of an example laryngoscopy procedure being performed on a patient.

### DETAILED DESCRIPTION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entirety and to the same extent as if each reference was individually incorporated by reference.

### EXAMPLE SYSTEM

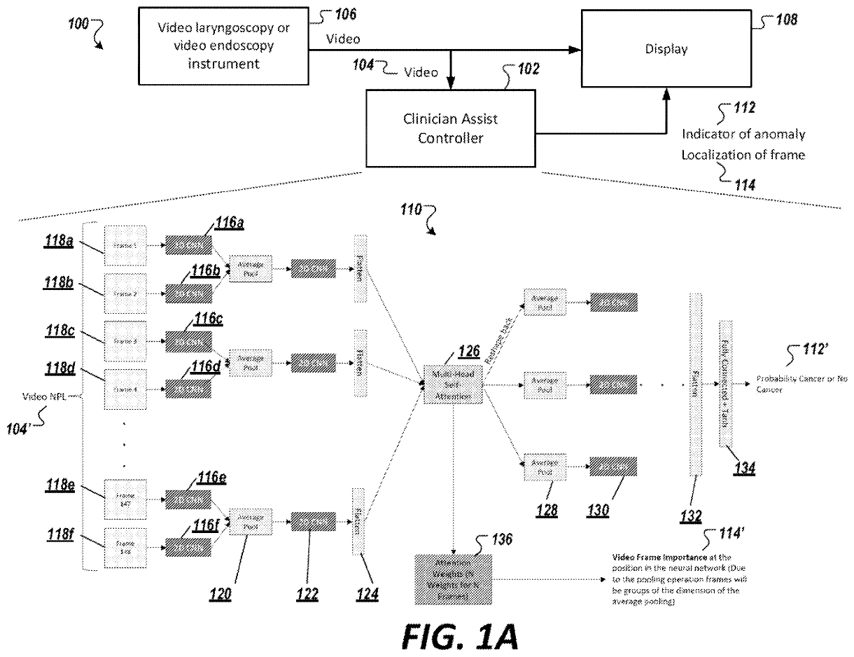

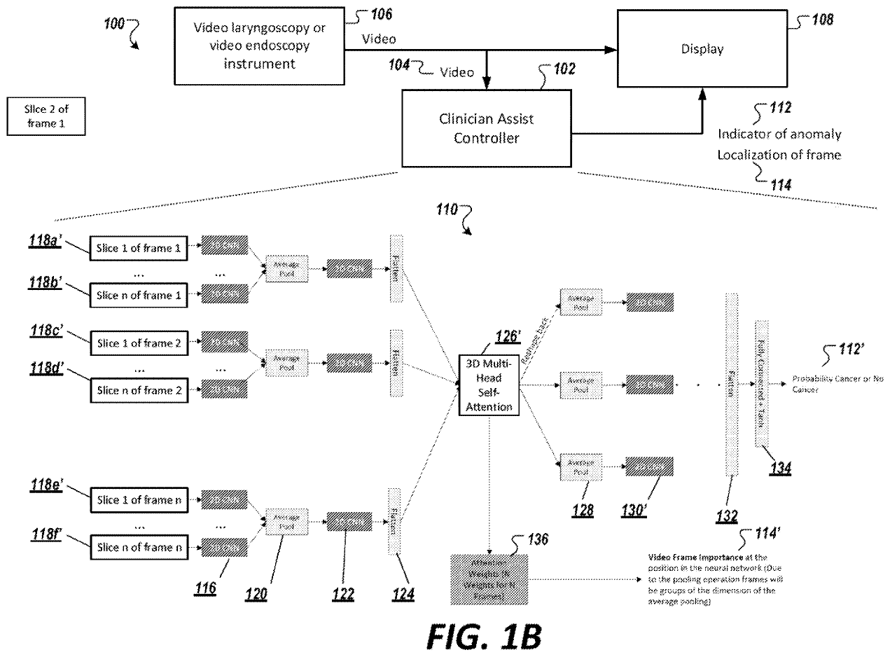

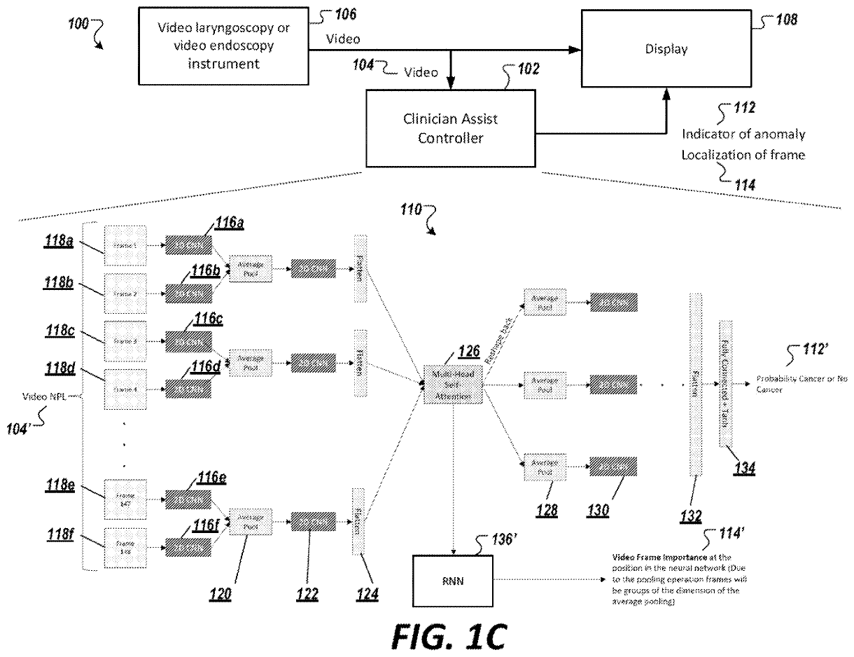

FIG. 1A shows an example system 100 comprising a clinical-assist system 102 (shown as “Clinician-assist controller” 102) for endoscopic or laryngoscopic video, e.g., Video Nasopharyngolaryngoscopy (NPL), that operates on video input 104 from an endoscopic or laryngoscopic instrument 106 to provide clinician assisted output, in accordance with an illustrative embodiment.

In the example shown in FIG. 1A, the endoscopic or laryngoscopic instrument 106 couples to a display 108 to display the acquired endoscopic or laryngoscopic video 104 to a clinician during a scan. The video frames of the video input are provided as direct input into clinician-assist system 102 comprising a trained neural network 110 to provide an output an indicator 112 of the presence of an anomaly, e.g., lesion, nodules, and/or vasculatures, as an estimate of disease and a localization indicator 114 of the video frame(s) contributing to that indication.

In FIG. 1A, the trained neural network 110 is a transformer configured to include a set of 2D CNN layers 116 (shown as 116a, 116b, 116c, 116d, 116e, 116f) that is configured to receive one or more frames 118 (shown as “Frame 1” 118a, “Frame 2” 118b, “Frame 3” 118c, “Frame 4” 118d, . . . , “Frame 147” 118e, “Frame 148” 118f) of the endoscopic or laryngoscopic video 104 (shown as “Video NPL” 104′). The transformer includes (i) a set of encoding layers that process an input tokens, herein, video frames, iteratively one layer after another and (ii) a set of decoding layers that iteratively process the encoder's output as well as the decoder output's tokens so far.

In FIG. 1A, the encoder layers include 2D CNN layers 116 coupled to average pool layer 120 that is then couples to second 2D CNN layer 122 that couples to a flatten layer 124 that couples to a multi-head self-attention module 126. The decoder layers include a set of one or more average pool layers 128 coupled to a 2D CNN layer 130 that connect to a flatten layer 132 and a fully connected layer +Tanh 134 to output a probability value or score for the anomaly 112 (shown as 112′) as the presence or non-presence of an anomaly, e.g., a cancer. The 2D CNN layers (e.g., 116, 122) extract features across each image frame within the video separately.

The multi-head self-attention module 126 is configured to allow the model to jointly attend to information from different representation subspaces, which in this instance, from across the different frames. In some embodiments, the multi-head self-attention module 126 is a multi-head dot-product self-attention module that can extract dependencies between frames to allow information, e.g., relating to lesions, nodules, vasculatures, in one frame to be considered in conjunction with other frames. An example of a multi-head dot-product self attention module is described in Vaswani, A., Shazeer, N., Parmar, N., Uszkoreit, J., Jones, L., Gomez, A., Kaiser, u., & Polosukhin, I. (2017), “Attention is All you Need,” In Advances in Neural Information Processing Systems. Curran Associates, Inc., which is incorporated by reference herein.

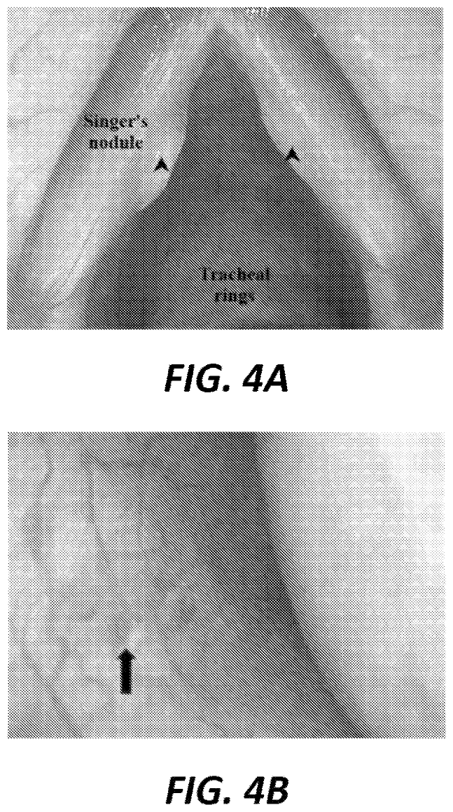

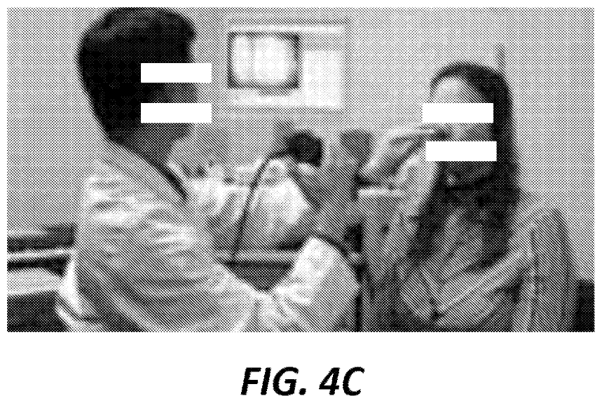

FIGS. 4A-4B show examples of lesions and nodules that may be found in neck, throat, or head region of a subject or patient. FIG. 4C shows a photo of a laryngoscopy procedure being performed on a patient. In FIG. 4C, the laryngoscopic or endoscopic instrument is inserted into the nose or mouth of a patient to image the structures therein. The image/video is presented in a display, which is used for the evaluation and also for guiding the instrument through the body spaces.

The multi-head self-attention module 126 operates with an attention weight module 136 that provides an output value of a position in the neural network contributing to the probability score. Put another way, the extraction of the frames that are most important to the classification, i.e., extracted learned attention weights, can be used to identify the frames the CNN gave the most attention to a given classification of indicators of cancer or no cancer. These frames associated with the weights could then be extracted and outputted to the clinician to provide the output indicator further examined. Multi-Head self-attention module 126 is configured to generate N attention weights for each head. The weights represent the relative importance of each frame within the video. Ideally, frames that are more relevant to the prediction of cancer will have higher attention weights. Frames with the highest attention weights can be further examined to understand what the model is using to make a given prediction of cancer or no cancer. For each frame (N Frames) input to the self-attention, the attention weight module 136 is configured to extract a single scalar weight (a1, a2, . . . , aN), which would, after training, represent its importance to the overall network.

In some embodiments, in the encoder layers of the transformers, each frame is associated with a 2D CNN and thus a position to the weights of neural network (e.g., 2D CNN) can be employed to provide an index to the frame of interest. The output value, as a localization indicator 114 (shown as 114′), is thus a set of video frame importance values that allows the identification of one or more video frames predominately contributing to the probability score of a disease indicator.

While the output of the trained neural network 110 is a set of visual indicators of an anomaly in the endoscopic or laryngoscopic video, e.g., Video Nasopharyngolaryngoscopy (NPL) to be used in the diagnostic, e.g., of cancer, the output itself is not a cancer diagnostic. And while the transformer NN is configured to provide an estimation or probability score for the presence or non-presence of a disease, the presence or non-presence indicator itself is based on the estimation of the presence or non-presence of lesions, nodules, or vasculatures in the video frames as a proxy for a disease. The output of the trained neural network 110 thus call attention to a clinician to identify the lesions, nodules, or vasculatures for subsequent evaluation in the diagnostic of a disease.

While the example shown in FIG. 1A shows 2D CNN (e.g., in layers 116, 122), the system 100 can be implemented with 3D data input (FIG. 1B). With 3D input, e.g., 3D computed tomography data having a volume composing of multiple layers across multiple frame, the processing of the frames can be performed independently similarly described in FIG. 1A. The multi-head self attention 126 (shown as 126′) becomes a point of aggregation of the information across the different layers and different frames. The multi-head self attention 126′ would employ 3D convolution to process N frame together, where N is set as a hyperparameter. In FIG. 1B, the CT volume is individually provided for each layer for a given frame into the 2D CNN (116), shown as 118a′ 118f′.

While the example shown in FIG. 1A shows a self-attention module, the system 100 can be employed with 2D CNN+RNN (FIG. 1C). With the 2D CNN+RNN, the system could perform feature extractions across frames and then employ the RNN, or a variant (e.g., LSTM or GRU) to process the dependencies between the frames.

### Example Implementation of Multi-Head Self Attention Module and Attention Weight Module

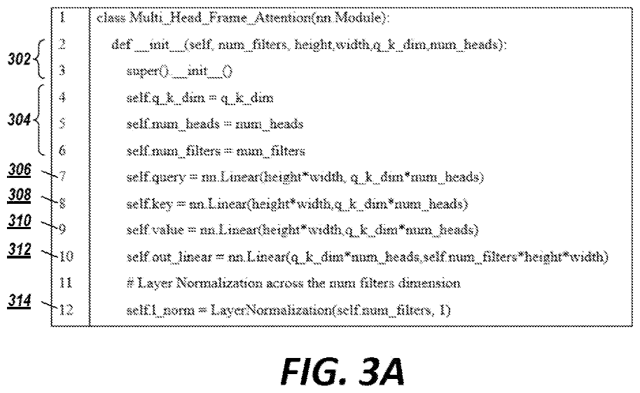

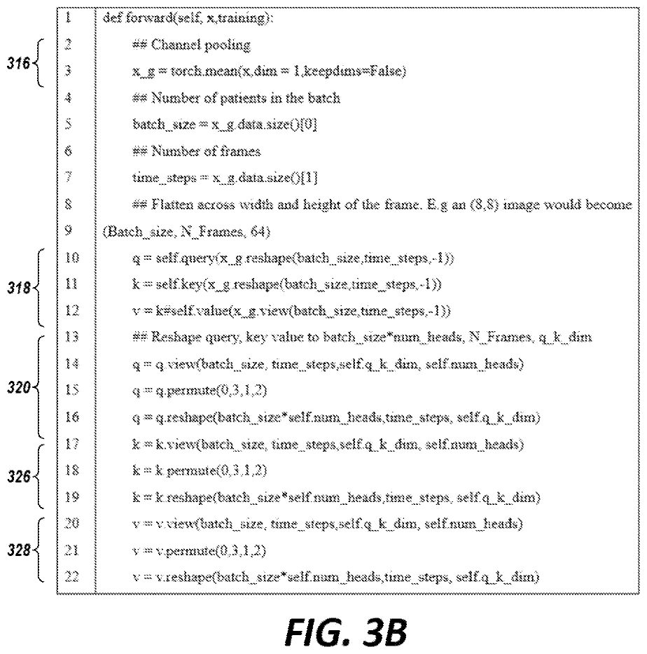

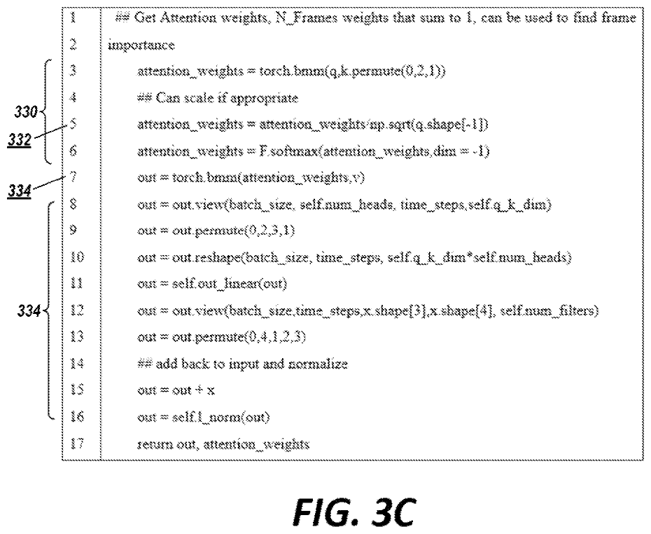

FIG. 3A provides computer instructions for a multi-head dot-product self attention module (e.g., 126). Table 1 provides an example set of parameters for the computer instructions.

TABLE 1

| Parameter | | |
|---|---|---|
| Name | Description | Example Value |
| q_k_dim | Total number of features for Query, | 8, 16, 32, etc. |
| | key, and value paramaters | |
| num_heads | Number of parallel attention heads | 1, 2, 3, 4, etc. |
| num_filters | Number of filters on the input | 3 for a RGB Image |
| height, width | Height and width of the image | 64 × 64, etc. |
| | frame (pixels) | |

In FIG. 3A, lines “2” and “3” (302) sets out the parameters for the multi-head dot-product self attention module. Lines “4,” “5,” “6” (304) define variables in the multi head frame_attention class. Line “7” (306) provides the query setting as the embeddings of shape for an unbatched input, based on the (i) height and weight of model and (ii) the number of attention headers and filters. Queries are compared against key-value pairs to produce the output. Line “8” (308) provides a key setting as the key embeddings of shapes for unbatched input based on the same 2 parameter sizes. Line “9” (310) provides a value setting as the value embeddings of shape for an unbatched input based on the same 2 parameter sizes. Line “10” (312) provides a filter setting as the filter embeddings of shape for an unbatched input based on (i) the number of attention headers and filters and (ii) the number of filters and the height and weight of the model. Line “12” (314) provides a layer normalization operation across the number of filter dimensions.

FIG. 3B shows computer instructions for calculating the query, key, and value embeddings during training and inference of the multi-head dot-product self attention module of FIG. 3A. In lines “2” and “3” (316), the input is first pooled across the channel dimension. The number of channels as input is determined by the number of filters learned by the convolution prior to the multi-head dot-product self attention module. Lines “10”-“12” (318) show the flattening of the frames across the width and height, e. g, a 64×64 image would flatten to size 4096, and then the subsequent forward pass through the query, key and value linear projections. Lines “13”-“16” (320) show the reshaping of the query to apply attention in parallel across num_heads by multiplying the batch dimension by the num_heads parameter. Lines “17”-“19” (326) and “20”-“22” (328) repeat the operation for the key and value projections.

FIG. 3C shows computer instructions for an attention weight module (e.g., 136). In Lines “3”-“6” (330) the attention weights are formed by calculating the dot product against the query and transposed key projections. The attention weights can then be scaled, as shown on Line “5” (332), before applying the softmax operation. In line “7” (334), the dot product between the attention weights and the value projection is calculated to calculate the weighted sum between each frame and the relative importance of the other image frames. In lines “8-16”0 (334), a linear projection is applied across the shape of the value projection multiplied by the number of attention heads to transform the output back to the original shape of the input. The output is then added back to the original inputs and the layer normalizion operation is applied. In line “16” (336), both the output and learned attention weights are returned the NN for further processing.

In some embodiments, the system 100 can be employed to provide a clinician assisted output that provides the presence or non-presence of anomalies such as lesions, nodules, and vasculatures, among others, in an endoscopic or laryngoscopic video, e.g., Video Nasopharyngolaryngoscopy (NPL) as evidence of a disease in such video. In some embodiments, the system 100 can be used to provide the presence or non-presence of anomalies such as lesions, nodules, and vasculatures, among others, as evidence of the presence or non-presence of oropharyngeal squamous cell carcinoma (OPSCC).

The quality of the trained neural network can depend on (i) the quality and size of the dataset, (ii) the ability of the attention mechanism to find the important attributes of cancer, rather than just noise within the dataset, (iii) the overall architecture and the attentions mechanisms place within the network, and (iv) the ability of the CNN to extract important features which indicate presence or non-presence of indicators of cancer or no cancer.

Several factors can affect the architecture of the NN and the attention mechanism. If the video NPL is deeper within the NN, for instance after the average pooling operation 128 shown in FIG. 1C, the video NPL will be “more compressed” due to average pooling, and the attention weights may represent larger groups, or a smoothed representation of the original N Frames that were input to the model. If the video NPL is close to the original input data, such as before the average pooling operation 120 shown in FIG. 1C, the attention weight module 136 can extract importance across each video frame input to the model.

The system 100, in being operable in evaluating mutiple frames across a video, can also be employed to evaluate indicator of a disease or a proxy of the disease in endoscopic or laryngoscopic video, e.g., Video Nasopharyngolaryngoscopy (NPL). In some embodiments, the system 100 can be employed to evaluate functional pathologies associated with neck, head, throat conditions that are exhibited across multiple frames. Examples of functional pathologies can include swallowing, breathing, phonation (e.g., act of vocal cord coming together), other body movement/vibration. Functional pathologies can also include the presence or indication of paralysis as well as aspiration, e.g., into the lungs or trachea in view of an action (e.g., swallowing, breathing, speaking, movements, etc.), as well as spitting. The exemplary system and method can be employed in conjunction with test protocols, e.g., to eavluat function a pathologies as the subject or patient is asked to swallow different types of liquids.

In some embodiments, the system 100 is configured to determine one or more frames associated with the presence of a disease state or condition, and wherein the (i) output score or the label and the (ii) an identifier associated with the one or more frames are presented in the endoscopic or laryngoscopic video. The label, e.g., can be encoded based on the output score.

For example, an output score greater than a threshold value of x can have the label “disease state potentially present” and an output score lower than the threshold value can have the label “disease state or condition not present.”

In some embodiments, the system 100 is configured to generate a single output score or label is generated for the input endoscopic or laryngoscopic video.

In other embodiments, the system 100 is configured to generate a unique output score or label for each frame or a portion of the frames of the input endoscopic or laryngoscopic video. In such embodiment, multiples of the trained AI model can be implemented to operate in parallel operation to process a suset of the frames of the input endoscopic or laryngoscopic video.

Alternative methods to localize lesions. Alternative to extracting the frames most important to the prediction, the system 100 can employ other algorithms, e.g., Grad-CAM described in R. R. Selvaraju, M. Cogswell, A. Das, R. Vedantam, D. Parikh and D. Batra, “Grad-CAM: Visual Explanations from Deep Networks via Gradient-Based Localization,” 2017 IEEE International Conference on Computer Vision (ICCV), Venice, Italy, 2017, pp. 618-626, doi: 10.1109/ICCV.2017.74, which is incorporated by reference herein. Additional examples/variations of the Grad-CAM algorithms can produce a saliency map to highlight the frames most important to the network's decision. The frames could then be extracted and further examined. Examples of such operations are described in A. Chattopadhay, A. Sarkar, P. Howlader and V. N. Balasubramanian, “Grad-CAM++: Generalized Gradient-Based Visual Explanations for Deep Convolutional Networks,” 2018 IEEE Winter Conference on Applications of Computer Vision (WACV), Lake Tahoe, NV, USA, 2018, pp. 839-847, doi: 10.1109/WACV.2018.00097 as well as Rachel Lea Draelos, & Lawrence Carin. (2021). Use HiResCAM instead of Grad-CAM for faithful explanations of convolutional neural networks, each of which is incorporated by reference herein.

Example System #2. As an alternative to 2D CNN, vision transformers can be utilized. In this architecture, instead of using convolutions for frame-wise feature extraction, the system can employ attention mechanisms and multi-layer-perceptron to classify the video. Another example of vision transformers is the SWIN transformer, which is configured to extract defined patches from the video or image frames and use those as the input to the transformer. Examples of vision transformers are provided in Alexey Dosovitskiy, Lucas Beyer, Alexander Kolesnikov, Dirk Weissenborn, Xiaohua Zhai, Thomas Unterthiner, Mostafa Dehghani, Matthias Minderer, Georg Heigold, Sylvain Gelly, Jakob Uszkoreit, & Neil Houlsby. (2021), “An Image is Worth 16×16 Words: Transformers for Image Recognition at Scale,” which is incorporated by reference herein. An example of the SWIN transformer is provided in Z. Liu, et al., “Swin Transformer: Hierarchical Vision Transformer using Shifted Windows,” in 2021 IEEE/CVF International Conference on Computer Vision (ICCV), Montreal, QC, Canada, 2021 pp. 9992-10002. doi: 10.1109/ICCV48922.2021.00986, which is incorporated by reference herein.

### EXAMPLE METHOD

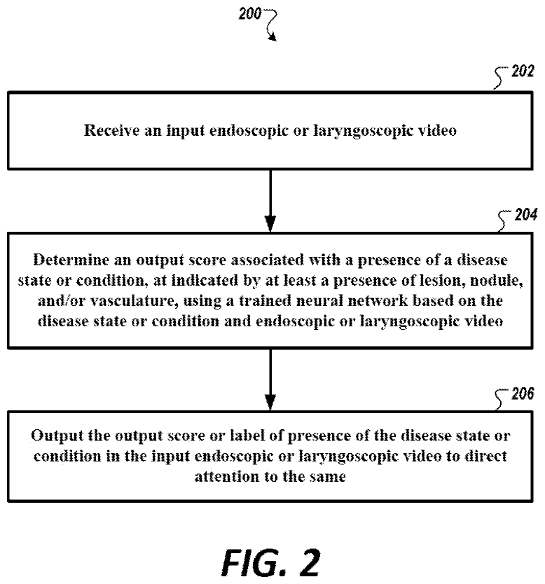

FIG. 2 shows a method 200 to perform operations in the clinical-assist system (e.g., 102) in accordance with an illustrative embodiment. In the example shown in FIG. 2, Method 200 includes receiving (202) an input endoscopic or laryngoscopic video (e.g., video endoscopy, video laryngoscopy, e.g., Video Nasopharyngolaryngoscopy). Method 200 then includes determining (204), via a processor, an output score associated with a presence of a disease state or condition, as indicated by at least one of a presence of lesion, nodule, and/or vasculature, using a trained neural network based on the endoscopic or laryngoscopic video. Method 200 then includes outputting (206) the output score or label derived from the same, wherein the output score or label is presented in the input endoscopic or laryngosc opic video to direct attention to the same.

In some embodiments, the trained neural network is configured to determine one or more frames associated with the presence of a disease state or condition, and wherein the (i) output score or the label and the (ii) an identifier associated with the one or more frames are presented in the endoscopic or laryngoscopic video.

In some embodiments, the output score associated with the presence of a disease state or condition includes a presence of a functional pathology.

In some embodiments, a unique output score or label is generated for each frame or a portion of the frames of the input endoscopic or laryngoscopic video.

In some embodiments, a single output score or label is generated for the input endoscopic or laryngoscopic video.

In some embodiments, the trained neural network was trained with a training data set comprising endoscopic or laryngoscopic video and an indication of a nodule as a ground truth.

In some embodiments, the trained neural network was trained with a training data set comprising endoscopic or laryngoscopic video and an indication of a lesion as a ground truth.

In some embodiments, the trained neural network was trained with a training data set comprising endoscopic or laryngoscopic video and an indication of vasculature as a ground truth.

### EXPERIMENTAL RESULTS AND EXAMPLES

A study was conducted that developed a system configured to identify indicators of disease in Nasopharyngolaryngoscopy exams with high accuracy. In the head and neck cancer domain, it is contemplated that the system could be employed to predict tumor location, assess response to therapy, screen for early recurrence, and diagnose treatment-associated toxicity. In the non-cancer domain, it is contemplated that the system can be used to assist clinicians in the clinician's diagnosis of benign lesions and functional pathology.

Training Data. NPL was provided by The Ohio State University Wexner Medical Center for 85 patients undergoing treatment or follow-up care for OPSCC from January 2019 to April 2022 (Table 2). Of the video data classified by clinicians, 17 patients showed no evidence of disease in the video taken post-treatment; 68 patients showed active signs of disease in the video, 3 of which were recurrence. Table 3 shows examples cross-valuation results observed in the study. A 2D Convolutional Neural Network (CNN) model was trained using the full video NPL data for each patient.

TABLE 2
Patient Characteristics

| | Patients with No | Patients with |
|---|---|---|
| | Evidence of Disease | Evidence of |
| Characteristic | at Scope | Disease at Scope |
| Number | 17 | 68 |
| Age, mean (range) | 60.88 (48-84) | 60.15 (29-77) |
| Sex | | |
| Male | 12 | 21 |
| Female | 5 | 47 |
| Recurrence of | 0 | 3 |
| disease at time of | | |
| scope | | |

TABLE 3
Cross-Validated Results

| | Cross- | |
|---|---|---|
| | Validated | |
| Metric | Result | Interpretation |
| Accuracy | 0.88 | proportion of patients correctly classified |
| Sensitivity | 0.90 | proportion of ‘active cancer’ patients |
| | | classified correctly |
| Specificity | 0.82 | proportion of ‘no evidence of disease’ |
| | | patients classified correctly |
| AUC | 0.82 | an aggregation of performance across all |
| | | thresholds |
| Precision | 0.94 | the probability a classification of ‘active |
| | | cancer’ is actually cancer |
| F1 Score | 0.89 | A balanced score measuring how well the model |
| | | identifies ‘active cancer’ without sacrificing |
| | | Precision. |

Results. Overall, the results show excellent performance in the ability of the ML model to predict active cancer or no evidence of disease in Video NPL, especially considering the small data size. While the results are encouraging, there is some variation in performance across folds. We expect this and overall performance to improve with a larger dataset.

Discussion. This is the first study showing potential use of recorded video NPL to predict the presence of cancer through machine learning. The presented model demonstrates the capabilities of the ML system. Further study is being undertaken on a larger dataset.

It is contemplated that the exemplary method and system can be used in diagnostic aid usable by both ENT and non-ENT experts (e.g., radiation oncologists or speech and language pathologists) who do not have the ability to biopsy, especially in post-treatment surveillance and identification of the unknown primary tumor for OPSCC. It is contemplated that the exemplary system and method can be employed as a clinician-assisted device for cancer detection across the larynx and pharynx, swallowing function, vocal cord paralysis, other benign laryngopharyngeal, and detection of clinical and subclinical toxicity from surgery and chemoradiation.

Example System. A 2D Convolutional Neural Network (CNN) model was trained similar to those described in FIG. 1A using the video NPL data. When a new, never-before seen video NPL was inputted to the model, it outputted a classification of ‘active cancer’ or ‘no evidence of disease’ as a proxy for determining indicators of cancer, such as nodules, legions, and/or vasculatures, among others. To address the long duration of the videos, every 20 frames were averaged to make videos a reasonable size for model fitting and validation. All videos were then zero-padded to an equal length of 148 frames before being passed for further processing by the model.

Example Training. To extract features across video frames, the study used a 2D Convolutional Neural Network (CNN) with 4 separate convolution phases, each using a kernel size of (3,3) followed by a rectified linear unit (ReLU) activation. The first 3 convolutions are then followed by an average pooling layer. To extract feature-wise dependencies between frames, we add a frame-wise dot product self-attention layer (Vaswani) after the second convolution. To extract frame-wise dependencies, self-attention is an alternative to using a recurrent neural network or 3D convolutional approach. In this approach, each frame can be attended to by itself as well as by every other frame, making self-attention capable of capturing longer-range dependencies. Each frame is flattened across the channel width and height dimensions before applying the self-attention mechanism. The CNN was built in PyTorch and trained for 100 epochs with the objective to minimize the cross-entropy loss. For training, the Adam optimizer was used with an initial learning rate of 0.001 and a batch size of 4. No data augmentation methods were used for this study. The model was trained using a single NVIDIA Volta V100 with 32 GB GPU memory, provided by the Ohio Supercomputer Center.

To validate generalization performance, the study used 5-fold stratified cross validation and aggregated results across folds. This means for each fold, roughly 68 patients' video NPL were used for training and 17 patients' video NPL were used to assess validation performance. The study used a cross validation strategy as, opposed to a train-test-validation split, every patient within the dataset can eventually be used in the validation set during the cross validation. As the dataset is small (85 patients) if the study used a train-test-validation split it would not have enough representation in a single validation set to adequately assess the model's ability to generalize.

Due to the small size of the dataset, the total number of features extracted is kept small, with the first convolution beginning with 4 output feature channels and doubling until the final 32 output feature channels for the last convolutional layer. From there, the extracted features are flattened and passed through two fully connected layers, with the final layer having one output neuron followed by a sigmoid activation for the classification of cancer or no cancer. The full model structure is shown in Supplemental FIG. 1.

Pytorch was used for all model building and training. We trained our CNN using binary cross entropy as the loss function with a fixed learning rate of 1e-3 over 100 epochs.

### Discussion

The current diagnosis of oropharyngeal squamous cell carcinoma (OPSCC) involves endoscopy, PET, and biopsy of suspicious lesions. However, identification of OPSCC is often difficult on endoscopy [1], [2]. Advancements that improve diagnostic capacity with minimal disruption of current clinical workflow would improve the identification of unknown primary tumors and local surveillance after treatment of OPSCC and other head and neck cancers (HNC).

Over the last decade, advancements in machine learning (ML) combined with collecting large medical datasets have resulted in increased research in the application of ML in medicine [3], including disease diagnosis, triage and prognostication, clinical decision-making, surgical planning, intra-operative assistance, and patient education [4]. Each year between 2005 and 2019 has had an estimated 61-fold increase in the number of papers applying ML to medicine. The applicability of ML in cancer diagnosis has seen a surge in evidential support with image analysis methods [5]-[10].

Similarly, research on ML applications in HNC has also increased [3]. Studies have used endoscopic data to detect cancer, but this has generally been multispectral narrow-band imaging (mNBI). No previous research has used video endoscopy.

Artificial Intelligence and Machine Learning. In addition to the machine learning techniques described above, the exemplary system and method may be implemented using other artificial intelligence and machine learning techniques or in combination therewith.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc., using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanH, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike a traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models: A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., presence of one feature in a class is unrelated to presence of any other features). NB classifiers are trained with a data set by computing the conditional probability distribution of each feature given label and applying Bayes' Theorem to compute conditional probability distribution of a label given an observation. NB classifiers are known in the art and are therefore not described in further detail herein.

A k-NN classifier is a supervised classification model that classifies new data points based on similarity measures (e.g., distance functions). k-NN classifiers are trained with a data set (also referred to herein as a “dataset”) to maximize or minimize an objective function, for example a measure of the k-NN classifier's performance, during training. This disclosure contemplates that any algorithm that finds the maximum or minimum of the objective function can be used. k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### EXAMPLE CONTROLLER

It should be appreciated that the logical operations described above, e.g., for the controller (e.g., 102) or other computing devices, can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

### Conclusion

Each and every feature described herein, and each and every combination of two or more of such features, is included within the scope of the present invention, provided that the features included in such a combination are not mutually inconsistent.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with certain embodiments and specific examples, it is not intended that the scope be limited to the particular embodiments set forth, as the embodiments herein are intended in all respects to be illustrative rather than restrictive.

The following patents, applications, and publications, as listed below and throughout this document, are hereby incorporated by reference in their entirety herein.

### REFERENCES

[1] Muto M, Nakane M, Katada C, et al. Squamous cell carcinoma in situ at oropharyngeal and hypopharyngeal mucosal sites. Cancer. 2004; 101(6):1375-1381. doi:10.1002/cncr.20482
[2] Mascharak S, Baird BJ, Holsinger FC. Detecting oropharyngeal carcinoma using multispectral, narrow-band imaging and machine learning. The Laryngoscope. 2018; 128(11):2514-2520. doi:10.1002/lary.27159
[3] Mahmood H, Shaban M, Rajpoot N, Khurram SA. Artificial Intelligence-based methods in head and neck cancer diagnosis: an overview. Br J Cancer. 2021; 124(12):1934-1940. doi:10.1038/s41416-021-01386-x
[4] Kuo RYL, Harrison CJ, Jones BE, Geoghegan L, Furniss D. Perspectives: A surgeon's guide to machine learning. Int J Surg. 2021;94:106133. doi:10.1016/j.ijsu.2021.106133
[5] Kourou K, Exarchos TP, Exarchos KP, Karamouzis MV, Fotiadis DI. Machine learning applications in cancer prognosis and prediction. Comput Struct Biotechnol J. 2015;13:8-17. doi:10.1016/j. csbj.2014.11.005
[6] Ehteshami Bejnordi B, Veta M, Johannes van Diest P, et al. Diagnostic Assessment of Deep Learning Algorithms for Detection of Lymph Node Metastases in Women With Breast Cancer. JAMA. 2017;318(22):2199-2210. doi:10.1001/jama.2017.14585
[7] Bera K, Schalper KA, Rimm DL, Velcheti V, Madabhushi A. Artificial intelligence in digital pathology—new tools for diagnosis and precision oncology. Nat Rev Clin Oncol. 2019;16(11):703-715. doi:10.1038/s41571-019-0252-y
[8] Zormpas-Petridis K, Failmezger H, Raza SEA, Roxanis I, Jamin Y, Yuan Y. Superpixel-Based Conditional Random Fields (SuperCRF): Incorporating Global and Local Context for Enhanced Deep Learning in Melanoma Histopathology. Front Oncol. 2019;9. Accessed Oct. 8, 2022. https://www.frontiersin.org/articles/10.3389/fonc.2019.01045
[9] Wang S, Yang DM, Rong R, et al. Artificial Intelligence in Lung Cancer Pathology Image Analysis. Cancers. 2019; 11(11):1673. doi:10.3390/cancers11111673
[10] Sirinukunwattana K, Ahmed Raza SE, Yee-Wah Tsang null, Snead DRJ, Cree IA, Rajpoot NM. Locality Sensitive Deep Learning for Detection and Classification of Nuclei in Routine Colon Cancer Histology Images. IEEE Trans Med Imaging. 2016;35(5):1196-1206. doi:10.1109/TMI.2016.2525803.

1. A method comprising:
receiving an input endoscopic or laryngoscopic video (e.g., video endoscopy, video laryngoscopy, e.g., Video Nasopharyngolaryngoscopy);
determining, via a processor, an output score associated with a presence of a disease state or condition, as indicated at least by a presence of lesion, nodule, and/or vasculature, using a trained neural network based on the endoscopic or laryngoscopic video; and
outputting (i) the output score or a label derived from the output score, wherein the output score or the label are presented in the input endoscopic or laryngoscopic video to assist clinical evaluation of a subject or patient.

2. The method of claim 1, wherein the trained neural network is configured to determine one or more frames associated with the presence of a disease state or condition, and wherein the (i) output score or the label and the (ii) an identifier associated with the one or more frames are presented in the endoscopic or laryngoscopic video.

3. The method of claim 1, wherein the output score associated with the presence of a disease state or condition includes a presence of a functional pathology.

4. The method of claim 1, wherein a unique output score or label is generated for each frame or a portion of the frames of the input endoscopic or laryngoscopic video.

5. The method of claim 1, wherein a single output score or label is generated for the input endoscopic or laryngoscopic video.

6. The method of claim 1, wherein the trained AI model comprises (i) CNN layers having CNN nodes that evaluate individual frames of the input endoscopic or laryngoscopic video and (ii) multi-head self attention module configured to evaluate the individual frame collectively.

7. The method of claim 6, wherein the trained AI model is a transformer.

8. The method of claim 1, wherein the trained AI model further includes an attention weight module or an RNN configured to determine importance of weight associated with the CNN layers, wherein the CNN nodes are individually mapped to the individual frames, the method further including:
identifying one or more frames contributing to the output score associated with the presence of a disease state or condition.

9. The method of claim 1, wherein a predefined number of frames of the input endoscopic or laryngoscopic video are combined to be provided to the trained neural network, and wherein each frame of the input endoscopic or laryngoscopic video is inputted into a respective CNN.

10. The method of claim 6, wherein the trained neural network includes a plurality of stages of 2D CNN, each separated by a rectified linear unit (ReLU) activation.

11. The method of claim 1, wherein the trained neural network was trained with a training data set comprising endoscopic or laryngoscopic video and an indication of a nodule as a ground truth.

12. The method of claim 1, wherein the trained neural network was trained with a training data set comprising endoscopic or laryngoscopic video and an indication of a lesion as a ground truth.

13. The method of claim 1, wherein the trained neural network was trained with a training data set comprising endoscopic or laryngoscopic video and an indication of vasculature as a ground truth.

14. The method of claim 1, wherein the trained neural network was trained with a training data set comprising endoscopic or laryngoscopic video and an indication of a presence of a disease.

15. The method of claim 1, wherein the trained neural network was trained with a training data set comprising endoscopic or laryngoscopic video and an indication of a presence of a functional pathology.

16. The method of claim 1, wherein the output score for lesion, nodule, and vasculature and an associated frame in the input endoscopic or laryngoscopic video are presented in real-time in association with the acquisition of the endoscopic or laryngoscopic video.

17. The method of claim 1, wherein the output score for lesion, nodule, and vasculature and an associated frame in the input endoscopic or laryngoscopic video are presented in following acquisition of the endoscopic or laryngoscopic video.

18. A system comprising:
an analysis system having a processor and a memory having instructions stored thereon, wherein execution of the instructions by the processor causes the processor to:
receive an input endoscopic or laryngoscopic video;
determine, via a processor, an output score associated with a disease state or condition, including a presence of lesion, nodule, and vasculature, using a trained neural network based on the disease state or condition and endoscopic or laryngoscopic video; and
output the output score, wherein the output score for lesion, nodule, and vasculature and an associated frame in the input endoscopic or laryngoscopic video are presented via a graphical user interface to direct attention to the same.

19. (canceled)

20. A non-transitory computer-readable medium having instructions stored thereon, wherein execution of the instructions by a processor causes the processor to:
receive an input endoscopic or laryngoscopic video;
determine an output score associated with a presence of a disease state or condition, as indicated at least by a presence of lesion, nodule, and/or vasculature, using a trained neural network based on the endoscopic or laryngoscopic video; and
output (i) the output score or a label derived from the output score, wherein the output score or the label are presented in the input endoscopic or laryngoscopic video to assist clinical evaluation of a subject or patient.

21. (canceled)

22. (canceled)

23. (canceled)
